(19) United States
(12) Patent Application Publication
(10) Pub. No.: US 2021/0307673 A1
(43) Pub. Date: Oct. 7, 2021

(54) SYSTEM AND METHOD FOR EARLY AND EFFICIENT PREDICTION OF EPILECTIC SEIZURES

(71) Applicant: University of Louisiana at Lafayette, Lafayette, LA (US)

(72) Inventors: Hisham Daoud, Lafayette, LA (US); Magdy Bayoumi, Lafayette, LA (US)

(21) Appl. No.: 17/210,786

(22) Filed: Mar. 24, 2021

Related U.S. Application Data
(60) Provisional application No. 63/001,616, filed on Mar. 30, 2020.

(51) Int. Cl. A61B 5/00; A61B 5/369; A61B 5/31; A61B 5/291

(52) U.S. Cl. CPC A61B 5/4094; A61B 5/7264; A61B 5/291; A61B 5/31; A61B 5/369

(57) ABSTRACT

A seizure prediction algorithm based on deep learning that integrates the feature extraction and classification processes into a single automated architecture is claimed herein. In the method, the computation complexity is reduced because there is no feature engineering. The method uses a novel algorithm for EEG channel selection in which the number of EEG channels is decreased to reduce the required memory for storing the data and parameters. In one or more embodiments, an IoT based framework for accurate epileptic seizure prediction system is disclosed.

### CROSS REFERENCE TO RELATED APPLICATIONS

This application claims priority to the U.S. Provisional Application No. 63/001,616 filed on Mar. 30, 2020.

### STATEMENT REGARDING FEDERALLY SPONSORED RESEARCH OR DEVELOPMENT

Not Applicable.

### REFERENCE TO A “SEQUENCE LISTING ,” A TABKE, OR A COMPUTER PROGRAM

Not Applicable.

### SUMMARY OF THE INVENTION

A seizure prediction algorithm based on deep learning that integrates the feature extraction and classification processes into a single automated architecture is claimed herein. In the method, the computation complexity is reduced because there is no feature engineering.

The method uses a novel algorithm for EEG channel selection in which the number of EEG channels is decreased to reduce the required memory for storing the data and parameters and to facilitate practical application as a wearable device

An internet of things (“IoT”) framework is disclosed, which connects the patient to the doctor and any chosen emergency service. The history of EEG recording is continuously uploaded to the cloud to be viewed by the doctor. If the seizure is predicted to occur in the future, an alarm is generated for the patient and sent to the doctor and any chosen emergency service to fully protect the patient from any associated risk.

### DESCRIPTION OF THE DRAWINGS

The drawings constitute a part of this specification and include exemplary embodiments of the SYSTEM AND METHOD FOR EARLY AND EFFICIENT PREDICTION OF EPILECTIC SEIZURES which may be embodied in various forms. It is to be understood that in some instances, various aspects of the invention may be shown exaggerated or enlarged to facilitate an understanding of the invention. Therefore, the drawings may not be to scale.

FIG. 1 shows brain states in a typical epileptic EEG recording.

FIG. 2 is block diagram of MLP based seizure predictor.

FIG. 3 is block diagram of DCNN+MLP based seizure predictor.

FIG. 4 is block diagram of DCNN+Bi-LSTM based seizure predictor.

FIG. 5(A) is block diagram of the semi-supervised DCAE+Bi-LSTM model, pre-training phase of DCAE to generate the reconstructed EEG signals from the latent space representation through unsupervised learning.

FIG. 5(B) is block diagram of the semi-supervised DCAE+Bi-LSTM model, pre-trained classifier that predicts seizures through supervised learning.

FIG. 6 is the architecture of the claimed MLP based classifier.

FIG. 7 is the architecture of the claimed DCNN front-end in DCNN based models.

FIG. 8 is basic LSTM cell.

FIG. 9 is unrolled bidirectional LSTM network.

FIG. 10 is the architecture of the claimed DCAE. C stands for convolution. P for pooling. D for deconvolution and U for upsampling layer.

FIG. 11 is the measured accuracy among three differing claimed algorithms.

FIG. 12 is the measured sensitivity among three different claimed algorithms.

FIG. 13 is the measured specificity among three claimed algorithms.

FIG. 14 is the measured false alarm rate among three claimed algorithms.

FIG. 15 is the measured training time on the test set among five claimed algorithms: MLP, DCNN+MLP, DCNN+Bi-LSTM. DCAE+Bi-LSTM and DCAE+Bi-LSTM+CS.

FIG. 16 shows one embodiment of the IoT based automatic seizure prediction.

FIG. 17 depicts the inventive algorithm for EEG Channel Selection.

FIG. 18 is a block diagram that depicts the convolution module implementation.

FIG. 19 shows one embodiment of a protocol for the IoT based seizure predication system.

FIG. 20 shows a line graph of the prediction history to be reviewed by the doctor and an example of SMS notification generated after seizure prediction.

### BACKGROUND

Epilepsy is one of the world's most common neurological diseases. Early prediction of the incoming seizures has a great influence on epileptic patients' life. Current treatment modalities of epilepsy are either drugs on which not all patients are responsive or surgery where not all brain areas are candidates for focal resection. There was a longtime belief that epileptic seizures are unpredictable. However, Electroencephalographic (EEG) signals have different patterns before seizure occurrence where these patterns appear hours before clinical symptoms. Finding a method that would lead to timely and accurate prediction of seizures and taking the appropriate action to prevent and provide the needed intervention would be a great way to save patients from Sudden Unexpected Death in Epilepsy (SUDEP).

Prediction of epileptic seizures has much more benefits in terms of prevention of any life-threatening injuries that may occur due to sudden seizure occurrence by taking the appropriate precautions like taking medicine, calling emergency or moving to a safe place. IoT has a pivotal role in applying seizure prediction techniques to wearable devices with network connectivity.

Brain electrical activity in epileptic patients is divided into four stages: preictal, ictal, postictal and interictal. Ictal is the stage where the seizures are taking place. Preictal refers to the brain activity before the seizure onset, while postictal is the stage after the seizure happened and finally the interictal is the seizure-free brain state rather than the previous states.

One aim of this invention is to accurately detect the preictal brain state and differentiate it from the prevailing interictal state as early as possible and make it suitable for real time. The features extraction and classification processes are combined into a single automated system. Raw EEG signal without any preprocessing is considered as the input to the system which further reduces the computations.

Four deep learning models are provided to extract the most discriminative features which enhance the classification accuracy and prediction time. This method takes advantage of the convolutional neural network in extracting the significant spatial features from different scalp positions and the recurrent neural network in expecting the incidence of seizures earlier than the current methods. From the machine learning perspective, seizure prediction is treated as a classification problem between preictal and interictal states. This automatic prediction method reduces the risks associated with the sudden occurrence of seizures.

The demand for smart healthcare system has been increasing with the increase in the population and the failure of traditional healthcare for fulfilling those needs. Epileptic seizure prediction based on edge-IoT is an example of smart healthcare that provides a better quality of life to epileptic patients. The IoT is a cyber-physical system that connects all real-world components. IoT facilitates remote doctor-patient monitoring and consultation.

In the prior art, there are various methods claimed to address the seizure prediction problem trying to reach high classification accuracy with early prediction. Because EEG signals are different across patients due to the variations in seizure type and location, most seizure prediction methods are patient-specific. In these prior art methods, supervised learning techniques are used through two main stages, which are feature extraction and classification between preictal states and interictal states. The prior art has categorized the feature extraction schemes in terms of localization into univariate and bivariate and in terms of linearity into linear and nonlinear. Multiple features are sometimes combined to capture the brain dynamics that ends up in dimensionality increase. The extracted features are used to train the classifier that could then be used for the analysis of new EEG recordings to predict the occurrence of the seizure by detecting the preictal state.

In the prior art, the extracted features are categorized into three main groups: time domain, frequency domain and nonlinear features. The prior art used some statistical measures like variance, skewness and kurtosis as time domain features. In some instances, the prior art calculated the spectral power of the EEG signals for frequency domain analysis. Some nonlinear features that are derived from the dynamic systems' theory were investigated such as Lyapunov exponent and dynamic similarity index. Based on the selected features, a prediction scheme that detects the preictal brain state is implemented. Most of the prior art claimed machine learning based prediction schemes like Support Vector Machine (SVM). SVM classifier is used in numerous studies to predict the epileptic seizures. SVMs achieved outstanding results over other types of classifiers in terms of specificity and sensitivity.

Deep learning algorithms achieved great success in multiple classification problems for various applications like computer vision and speech recognition. Some prior art utilized deep learning in the classification stage for seizure prediction problem. The prior art has applied multi-layer perceptron to the extracted features and used a convolutional neural network as a classifier that is applied on the extracted features from EEG data to predict seizures.

The main challenge of the previously methods is to determine the most discriminative features that best represent each class—that is, how to determine the significant features that help the classifier taking the right decision. The computation time and required storage needed to extract these features depends on the process complexity and is considered another challenge especially in real-time application. Moreover, the current seizure prediction methods are not suitable for wearable constrained IoT devices because of the high computation complexity and large required memory footprint due to multiple connected channels. All these restrictions are critical in IoT constrained devices.

Motivated by these challenges and due to the significance of the early and accurate seizure prediction, the current invention develops deep learning based seizure prediction algorithms that combine the feature extraction and classification stages into a single automated framework.

A novel patient-specific seizure prediction technique based on deep learning and applied to long-term scalp electroencephalogram (EEG) recordings is provided herein.

### DETAILED DESCRIPTION

The subject matter of the present invention is described with specificity herein to meet statutory requirements. However, the description itself is not intended to necessarily limit the scope of claims.

Epilepsy is defined according to the International League Against Epilepsy (ILAE) report, as a neurological brain disorder identified by the frequent occurrence of symptoms called epileptic seizure due to abnormal brain activities. Seizure's characteristics include loss of awareness or consciousness and disturbances of movement, sensation or other cognitive functions. The overall incident of epilepsy is 23-100 per 100,000. People at extremes of age are the most affected age group while the disease crests among young individuals in ages between 10 to 20 years old.

Epilepsy has a high disease burden where 50 million people worldwide have epilepsy and there are about two million new patients recorded every year. Up to 70% of the epileptic patients could be controlled by the Anti-Epileptic Drugs (AED) while the other 30% are uncontrollable.

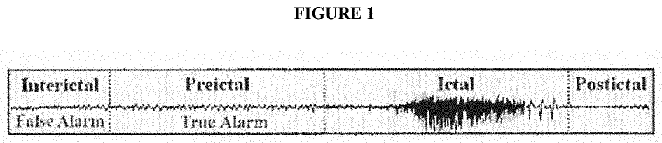

Electroencephalogram (EEG) is the electrical recording of the brain activities and is considered the most powerful diagnostic and analytical tool of epilepsy. Physicians classify the brain activity of the epileptic patients according to the EEG recordings into four states: preictal state, which is defined by the time period just before the seizure, ictal state which is during the seizure occurrence, postictal state that is assigned to the period after the seizure took place and finally the interictal state which refers to the period between seizures other than the previously mentioned states, these four states are illustrated in FIG. 1.

Due to unexpected seizure times, epilepsy has a strong psychological and social impact and classified as a life-threatening disease. Consequently, the prediction of epileptic seizures would greatly contribute to improving the quality of life of epileptic patients in many aspects, like raising an alarm before the occurrence of the seizure to provide enough time for taking proper action, developing new treatment methods and setting new strategies to better understand the nature of the disease. According to the above categorization of the epileptic patient's brain activities, the seizure prediction problem could he viewed as a classification task between the preictal and interictal brain states. An alarm is raised in case of detecting the preictal state among the predominant interictal states indicating a potential seizure is coming as shown in FIG. 1. The prediction time is the time before the seizure onset when the preictal state is detected.

This invention provides automatic extraction of the most important features by developing deep learning based algorithms without any preprocessing. Multi-Layer Perceptron is applied to the raw EEG recordings as a simple architecture of multiple trainable hidden layers, then Deep Convolutional Neural Network (DCNN) is used to learn the discriminative spatial features between interictal and preictal states.

In one embodiment, Bidirectional Long Short-Term Memory (Bi-LSTM) Recurrent Neural Network is concatenated to the DCNN to do the classification task. An Autoencoder (AE) based semi-supervised model is used and pre-trained using transfer learning technique to enhance the model optimization and converge faster. For the system to be suitable for real-time usage, computation complexity should he considered, therefore the invention provides a channel selection algorithm to select the best representing channels from the multi-channel EEG recording. The used testing method proves the robustness of the claimed algorithms over different seizures.

The invention provides four deep learning based models for the purpose of early and accurate seizure prediction taking into account the real-time operation. The seizure prediction problem is formulated as a classification task between interictal and preictal brain states, in which a true alarm is considered when the preictal state is detected within the predetermined preictal period as shown in FIG. 1.

Although there is a fair amount of prior art focused on seizure prediction, there is no standard duration for the preictal state. The preictal duration was chosen to be one hour before the seizure onset and interictal duration was chosen to be at least four hours before or after any seizure. Raw EEG data without any preprocessing and without handcrafted features extracting is used as the input to all the models. The discriminative features are learned automatically using the deep learning algorithms in order to reduce the overhead and speed up the classification task. Due to the limited number of seizures for each patient, there is an imbalance between preictal and interictal samples. Obviously, the number of interictal samples is much larger than the number of preictal samples, and the classifiers tend to be more accurate toward the class with the larger number of training samples.

This inventive method selects the number of interictal samples to be equal to the number of preictal samples to make the data balanced. The EEG signals are divided to non-overlapping five seconds segments, each segment is considered as a training batch.

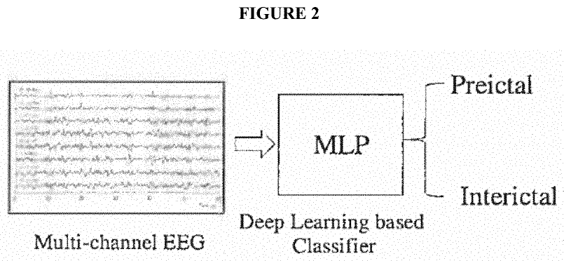

In the first model, Multi-layer Perceptron (MLP), a simple deep neural network, is trained on the selected patients to learn the network parameters that are able to do the classification task. The block diagram of the model is shown in FIG. 2.

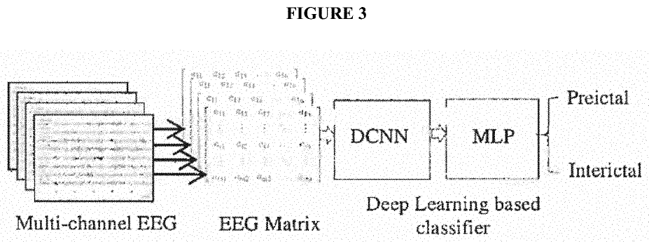

To enhance the classification accuracy, the inventive method provides the second model that relies on Deep Convolutional Neural Network (DCNN) which extracts the spatial features from different electrodes' locations and uses MLP for the classification task as illustrated in FIG. 3. In order to use DCNN, EEG data is represented by a matrix with one dimension is the number of channels and the other dimension is the time steps.

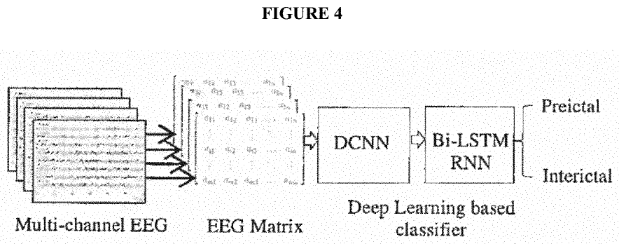

In the third model, DCNN is utilized and concatenated with a Bidirectional Long Short-Term Memory (Bi-LSTM) Network as the model back-end to do the classification as shown in FIG. 4. LSTM networks are known for their excellence in learning temporal features while maintaining long-time sequences dependencies which helps in early prediction. Prediction problems are handled better using Bi-LSTM as it uses information from both previous and next time instances.

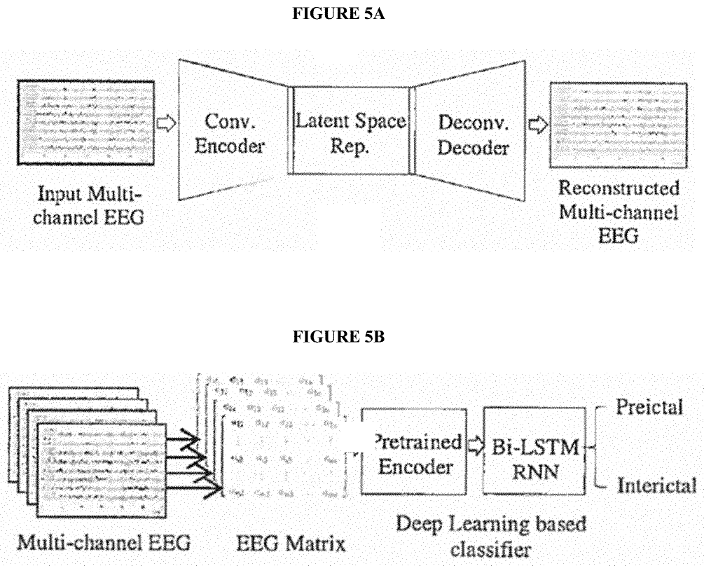

For the sake of training time reduction, the fourth model implements Deep convolutional Autoencoder (DCAE) architecture. In DCAE, the method pre-trained the model front-end, DCNN, in an unsupervised manner. Then, the training process is launched with some initial values that will help the network to converge faster and enhance the network optimization which in turn reduce the training time and increase the accuracy. Transfer learning approach is used to train the DCAE to improve the generalization across different seizures for the same patient. After training the AE, the trained encoder is connected to Bi-LSTM network for classification. FIG. 5 illustrates the two parts of the DCAE model. This method provides a channel selection algorithm to reduce the number of EEG channels which successively reduce the computation complexity and allocated memory making the system suitable for real-time application.

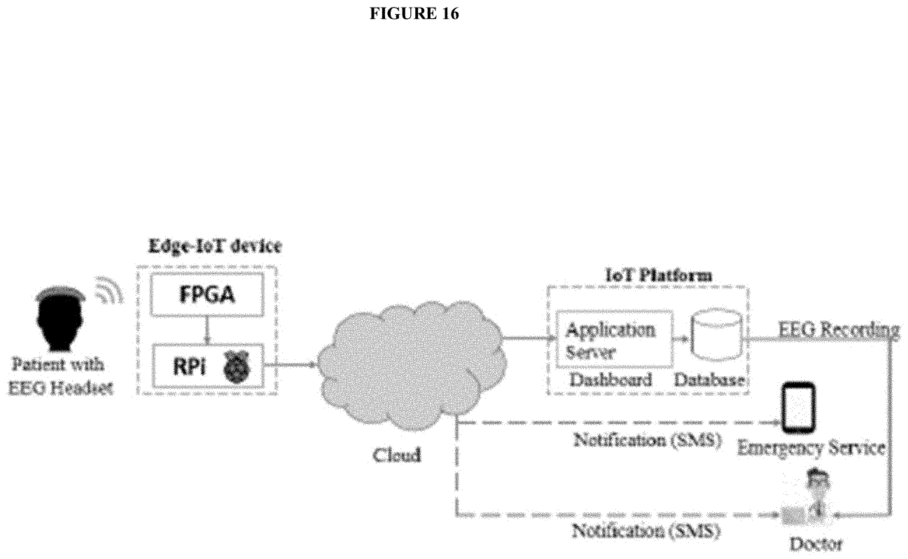

In one or more embodiments, an IoT framework for accurate epileptic seizure prediction that is suitable for real-time operation is incorporated. The framework is depicted in FIG. 16. The EEG recording is measured via a wireless headset that is commercially available. Then the EEG data is transmitted to the field-programmable gate array (“FPGA”), where the deep learning algorithm is embedded. The model takes raw EEG signals without any kind of preprocessing as its input. The FPGA sends the prediction results as well as the recorded EEG data to the processor. After predicting the seizure status at the edge, the positive prediction result raises an alarm to the patient and sends a notification to the doctor and any chosen emergency service via SMS. The continuous EEG recording is uploaded to the cloud to be viewed and evaluated by the doctor.

### EXAMPLE 1

The method was tested by training the proposed models and evaluating their performance on the CHB-MIT EEG dataset recorded at Children's Hospital Boston, which is publicly available. The dataset composed of long-term scalp EEG data for 22 pediatric subjects with intractable seizures and one recording with missing data. The recordings were taken during several days after anti-seizure medication withdrawal to characterize their seizures and evaluate their candidacy for surgical intervention. Most cases have EEG recordings from surface electrodes of 23 channels in accordance with the International 10-20 system. The sampling rate of the acquired EEG signals is 256 samples per second with 16-bit resolution. There are some variations in many factors between all subjects such as interictal period, preictal period, number of channels, and recording continuity. Therefore, eight subjects were chosen such that the pre-determined interictal and preictal periods are satisfied, the recordings are not interrupted and the full channels' recordings are available. Table 1 summarizes the details about the EEG recordings used in the experiments.

TABLE 1

| | | NUMBER OF | TOTLA SEIZURE |
|---|---|---|---|
| Case | ID-GENDER-AGE | SEIZURES | TIME (S) |
| 1 | 1-F-11 | 7 | 442 |
| 2 | 3-F-14 | 7 | 402 |
| 3 | 7-F-14.5 | 3 | 325 |
| 4 | 9-F-10 | 4 | 276 |
| 5 | 10-M-3 | 7 | 447 |
| 6 | 20-F-6 | 8 | 294 |
| 7 | 21-F-13 | 4 | 199 |
| 8 | 22-F-9 | 3 | 204 |

Multilayer Perceptron (MLP) is considered one of the most widely used artificial neural network (ANN). MLP consists usually of three successive layers, called: input layer, hidden layers, and output layer. Deep ANNs are composed of multiple hidden layers that enable the network to learn the features better using the non-linear activation functions. The ANN idea is motivated by the structure of the human brain's neural system. A typical ANN is a buildup of connected units called neurons. These artificial neurons incorporate the received data and transmit it to the other associated neurons, much like the biological neurons in the brain. The output of a neuron in any ANN is computed by applying a linear or non-linear activation function to the weighted sum of the neurons' output in the preceding layer. When the ANN used as a classifier, the final output at the output layer indicates the appropriate predicted class of the corresponding input data.

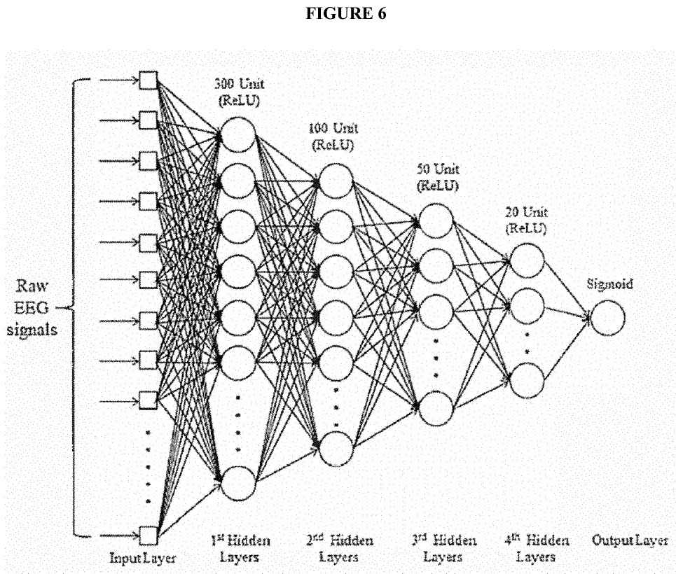

In the first claimed seizure prediction model, FIG. 2, the raw EEG is applied after segmentation to MLP with four hidden layers as depicted in FIG. 6. The number of units in each layer is 300, 100, 50, 20 starting from the first hidden layer to the fourth one. The total number of trainable parameters is 8,870,291 which is considered high due to the fully connected architecture. The model is trained with backpropagation and optimized using RMSprop algorithm. The loss function used is the binary cross entropy defined by (1).

l(y,ŷ)=−[y log(ŷ)+(1−y)log(1−ŷ)] (1)

where ŷ and y are the desired output and the calculated output respectively and l (y, ŷ) is the loss function.

Rectifier Linear Unit (ReLU) activation function, as defined by (2), is used across the hidden layers to add nonlinearity and to ensure robustness against noise in the input data.

$$f ( x ) = \{ \begin{matrix}x if x > 0 \\ 0 if x < 0\end{matrix} \qquad ( 2 )$$

where x is the sum of the weighted input signals f (x) is the ReLU activation function.

Sigmoid activation function (3) is selected for the output layer to predict the input data class.

$${p}_{i} = \frac{1}{1 + {e}^{- {x}_{i}}} \qquad ( 3 )$$

where xi is the sum of the weighted input signals and pi is the probability of the input example being preictal.

Convolutional Neural networks (CNNs) have shown great success in different pattern recognition and computer vision applications. This is due to the ability of CNN to automatically extract significant spatial features that best represents the data from its raw form without any preprocessing and without any human decision in selecting these features.

The sparse connectivity and parameter sharing of CNN give it high superiority regarding the memory footprint as it requires much less memory to store the sparse weights. The equivariant representation property of the CNN increases the detection accuracy of a pattern when it exists in a different location across the image. A typical CNN formed of three types of layers: convolution layer, pooling layer and fully connected layer. The convolution layer is used to generate the feature map by applying filters with trainable weights to the input data. This feature map is then down-sampled by applying the pooling layer to reduce the features' dimension and therefore the computational complexity. Finally, the fully connected layer is applied to all the preceding layer's output to generate the one-dimensional feature vector. CNN is used as a feature extractor to replace the complex feature engineering used in prior art.

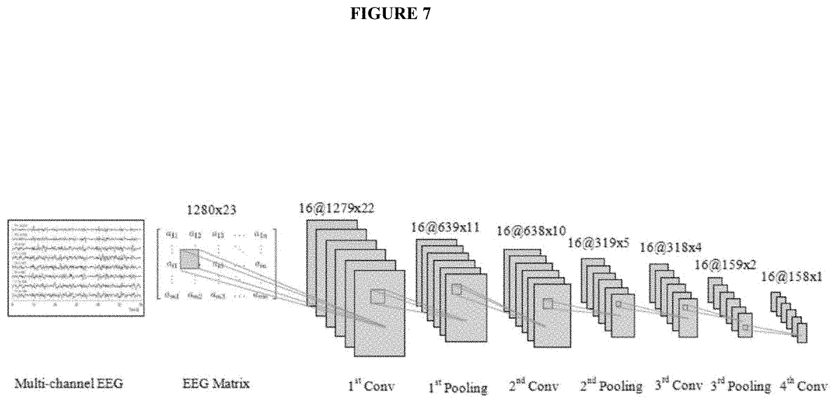

The claimed DCNN architecture model is shown in FIG. 7, in which the EEG segment is converted into a 2D matrix to be suitable for the DCNN. The architecture consists of four convolutional layers and three maximum pooling layers inter-changeably. The number of kernels in each convolution layer is 32 with kernel size of 3×2 to cover the non-square matrix of EEG data. The maximum pooling layers have pool size of 2×2. ReLU activation function is used across all the convolutional layers. Batch Normalization technique is used to improve the training speed and reduce overfitting through adding some noise to each layer's activation.

The Batch Normalization Transform is defined as:

$${BN}_{γ , β} ( {x}_{i} ) = γ \frac{{x}_{i} - {μ}_{B}}{\sqrt{{σ}_{B}^{2} + ϵ}} + β \qquad ( 4 )$$

where xᵢ is the vector to be normalized in a mini-batch B={x₁, x₂, . . . , xₘ}. μ_{B} and σ_{B}² are the mean and variance of the current mini-batch of xi, respectively. ∈ is a constant added to the mini-batch variance for numerical stability. γ and β are learned parameters used to scale and shift the normalized value respectively.

The claimed DCNN architecture is used as the front-end feature extractor in the three claimed models in FIGS. 3, 4, 5(b) which helps in spatial feature extraction from the different electrodes position on the scalp. The number of trainable parameters is drastically decreased when employing DCNN due to the weight sharing property. The number of trainable parameters in the second model, DCNN+MLP, is almost 520K, while in the third and fourth model, DCNN+Bi-LSTM and DCAE+Bi-LSTM, the number of trainable parameters is almost 28K.

Recurrent neural network (RNN) is a type of neural network that can maintain state along the sequential inputs. It can process a temporal sequence of data depending on the processing done on the previous sequences. This property of RNN makes it suitable for applications like prediction of time series data. The typical architecture of RNN is trained using backpropagation through time (BPTT) which has some drawbacks like exploding and vanishing gradients and information morphing.

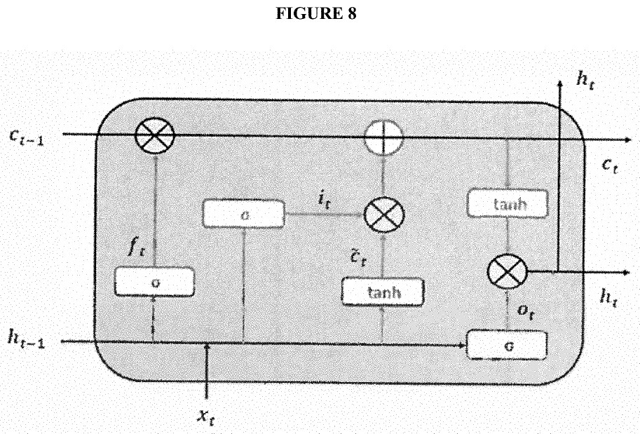

Long Short Term Memory Networks (LSTMs) are a type of RNN, implemented to overcome the problems of basic RNN. LSTMs are able to solve the problem of vanishing gradient by maintaining the gradient values during the training process and backpropagate it through layer and time, thus LSTM has the capability of learning long-term dependencies. LSTM cell, as shown in FIG. 8 consists of three controlling gates that could store or forget the previous state and use or discard the current state. Any LSTM cell computes two states at each time step: a cell state (c) that could be maintained for long time steps and a hidden state (h) that is the new output of the cell at each time step. The mathematical expressions governing the cell gates' operation are defined as follows:

fₜ=σ(W_{fh}hₜ₋₁+W_{fχ}χₜb_{f}) (5)

iₜ=σ(Wᵢₕhₜ₋₁+W_{iχ}χₜbᵢ) (6)

oₜ=σ(Wₒₕhₜ₋₁+W_{oχ}χₜbₒ) (7)

\tilde{c}ₜ=tanh(W_{ch}hₜ₋₁+W_{cχ}χₜb_{c}) (8)

cₜ=fₜ○cₜ₋₁+iₜ○\tilde{c}ₜ (9)

hₜ=oₜ○tanh(Cₜ) (10)

where xₜ is the input at time t, cₜ and hₜ are the cell state and the hidden state at time t respectively. W and b denote weights and biases parameters respectively. a is the sigmoid function and o is the Hadamard product operator. c,t is a candidate for updating ct through the input gate.

The input gate iₜ decides whether to update the cell with a new cell state cₜ, while the forget gate fₜ decides what to keep or forget from the previous cell state and finally the output gate oₜ decides how much information to be passed to the next cell.

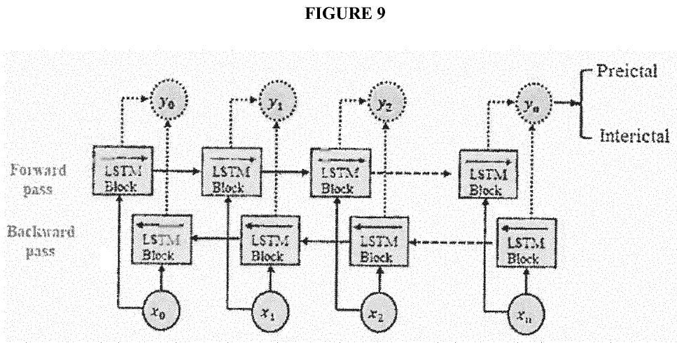

Instead of using LSTM as the classifier, a Bidirectional-LSTM (Bi-LSTM) network is used, in which each LSTM block is replaced by two blocks that process temporal sequence simultaneously in two opposite directions as depicted in FIG. 9. In the forward pass block, the feature vector generated from the DCNN is processed starting from its first-time instance to the end, while the backward pass block processes the same segment in the reverse order. The network output at each time step is the combined outputs of the two blocks at this time step. In addition to the previous context processing in standard LSTM, Bi-LSTM processes the future context which enhances the prediction results. Using Bi-LSTM as a classifier enhances the prediction accuracy through extracting the important temporal features in addition to the spatial features extracted by the DCNN.

Bi-LSTM is used in two claimed models in FIGS. 4, 5(b), as the back-end classifier that works on the feature vector generated by DCNN. The claimed network consists of a single bidirectional layer that predicts the class label at the last time instance after processing all the EEG segments as shown in FIG. 9. The number of units, dimensionality of the output space, is 20. Dropout regularization technique is utilized to avoid overfitting. The dropout is applied to the input and the recurrent state with factor of 10% and 50% respectively. The sigmoid activation function is used for prediction of the EEG segment's class and RMSprop is selected for optimization.

Autoencoders (AEs) are unsupervised neural networks whose target is to find a lower dimensional representation of the input data. This technique has many applications like data compression, dimensionality reduction, visualizing high dimensional data and removing noise from the input data. The AE network has two main parts namely, encoder and decoder. The encoder compresses the high dimensional input data into lower dimensional representation called latent space representation or bottleneck and the decoder is retrieving the data back to its original dimension. The simple AE uses fully connected layers for the encoder and decoder. The aim is to learn the parameters that minimize the cost function which expresses the difference between the original data and the retrieved one. Deep Convolutional Autoencoder (DCAE) replaces the fully connected layers in the simple AE with convolution layers.

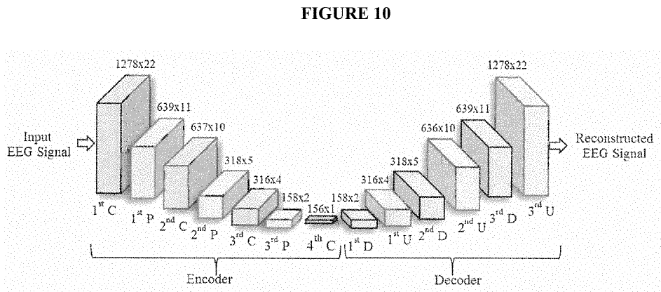

Due to the limited EEG dataset for each patient, an unsupervised training algorithm is developed using DCAE as shown in FIG. 5(a). The claimed architecture of the DCAE model is depicted in FIG. 10. It uses the same claimed DCNN model as an encoder and added the decoder network to build the DCAE. Unsupervised learning is deployed using transfer learning technique by training the DCAE on all the selected patients' data (not patient-specific). Transfer learning helps to obtain better generalization and enhance the optimization of the prediction model and therefore reducing the training time.

In the DCAE, FIG. 10 the encoder part consists of convolution and pooling layers interchangeably, while in the decoder part, the deconvolution and upsampling layers are used to reconstruct the original EEG segment. The encoder output is the latent space representation which is low dimensional features that best represent the EEG input segment. On the other hand, the decoder output is the reconstructed version of the original input. The learned encoder parameters are saved to be used later for training the prediction model in FIG. 5(b) allowing the training process to have a good start point instead of random initialization of the parameters which reduces the training time drastically.

Training of the DCAE is done using unlabeled EEG segments (balanced data of preictal and interictal segments) of all the selected patients. ReLU activation function is used across all the convolutional layers. Batch Normalization technique is used to improve the training speed and to reduce overfitting. The DCAE is optimized using RMSprop optimizer. The mean square error is utilized as the cost function and is defined as

$$J ( θ ) = \frac{1}{2 m} {\sum }_{i = 1}^{m} {( {{x}^{'}}^{( i )} - {x}^{( i )} )}^{2} \qquad ( 11 )$$

where x⁽ⁱ⁾ is the input EEG signal and \acute{x}⁽ⁱ⁾ is the reconstructed EEG signal. m is the number of training examples and θ is the parameters being learned.

After DCAE training, the pre-trained encoder is used as a front-end of the fourth claimed model, DCAE+Bi-LSTM, as shown in FIG. 5(b) while the back-end is Bi-LSTM network. The same network architecture of the DCNN and Bi-LSTM that is used in the third model (DCNN+Bi-LSTM) is used here. Training of this model is done in a supervised manner to predict the patient-specific seizure onset. Since both unsupervised and supervised learning algorithms were used, this model is considered a semi-supervised learning model.

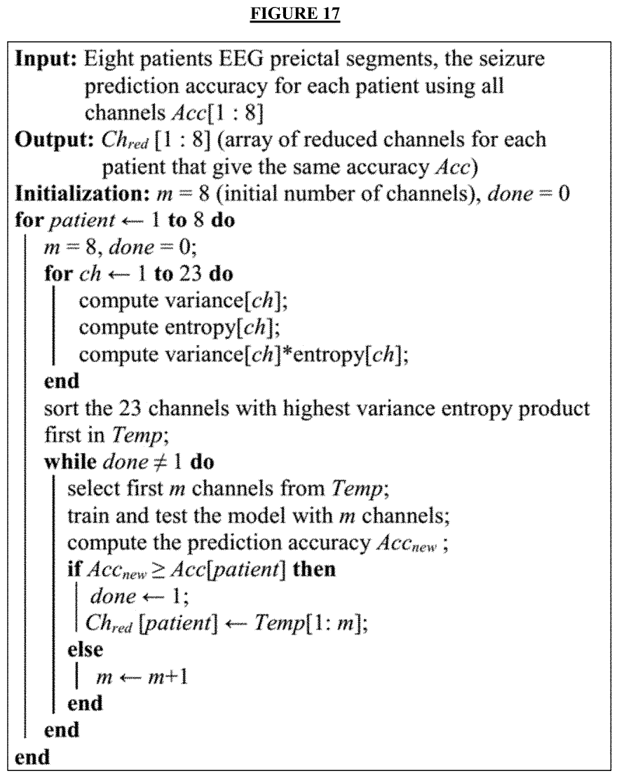

An EEG channel selection algorithm is introduced to select the most important and informative EEG channels related to the problem. Decreasing the number of channels helps with reducing the features' dimension, the computation load and the required memory for the model to be suitable for real-time application. The claimed channel selection algorithm is explained in FIG. 17. The algorithm is provided with the EEG preictal segments for each patient and the measured prediction accuracy by running the fourth model, DCAE+Bi-LSTM using all channels. On the other hand, the algorithm will output the reduced channels that give the same accuracy by omitting redundant or irrelevant channels.

First, the statistical variance defined by (12) and the entropy defined by (13) for all the available channels (23 channels) of the preictal segments are computed. The channels with highest variance entropy product that provide the same given prediction accuracy are selected. This is done through an iterative process by training the model on the reduced channels over each iteration. The variance is estimated as

$${σ}^{2} ( {X}_{c} ) = \frac{1}{N} {\sum }_{i = 1}^{N} {( {x}_{c} ( i ) - {μ}_{c} )}^{2} \qquad ( 12 )$$

where X_{c}, μ_{c} and N are the EEG data after normalization, mean and number of samples of channel c, respectively. The entropy of channel c is calculated as

H(X)=−Σₜ₌₁^{N}p(x_{c}(f))log₂ p(x_{c}(f)) (13)

where p(x_{c}(i)) is the probability mass function of the channel c having N samples.

In the channel selection algorithm, the channels with the highest variance entropy product are chosen to maximize both. This results in selection of the channel that has a high variance during the preictal interval and also provide the largest amount of information.

In order to overcome the problem of the imbalanced dataset, the number of interictal segments is chosen to be equal to the available number of preictal segments during the training process. The interictal segments were selected at random from the overall interictal samples. To ensure robustness and generality of the claimed models, use the Leave-one-out cross validation (LOOCV) technique as the evaluation method for all of the claimed models. In LOOCV, the training is done N separate times, where N is the number of seizures for a specific patient. Each time, all seizures are involved in the training process except one seizure on which the testing is applied. The process is then repeated by changing the seizure under test. By using this method, the testing covers all the seizures and the tested seizures are unseen during the training. The performance for one patient is the average across N trials and the overall performance is the average across all patients.

80% of the training data is assigned to the training set while 20% is assigned to the validation set over which the hyperparameters are updated and the model is optimized.

The performance of the models is evaluated by calculating some measures such as sensitivity, specificity, and accuracy on the test data. These measures are averaged across all patients. The prediction time of each model is recorded at the time of first preictal segment detection. The evaluation measures are defined as follows:

$$\begin{matrix}Sensitivity = \frac{TP}{TP + FN} & ( 14 ) \\ Specificity = \frac{TN}{TN + FP} & ( 15 ) \\ Accuracy = \frac{TP + TN}{TP + TN + FP + FN} & ( 16 )\end{matrix}$$

where TN, TP, FN and FP are the true negative, true positive, false negative and false positive respectively.

The claimed patient-specific models on the selected patients are evaluated by calculating some performance measures such as prediction accuracy, prediction time, sensitivity, specificity and false alarm per hour. The training time is also computed to evaluate the claimed channel selection algorithm. Table 2 shows the obtained values of these measures for the claimed four models which are MLP, DCNN+MLP, DCNN+Bi-LSTM, DCAE+Bi-LSTM. The fifth model, DCAE+Bi-LSTM+CS, is the same as the fourth one but with using the channel selection algorithm.

As could be noticed from Table 2, MLP has the worst accuracy, sensitivity, specificity and false alarm rate among the claimed models and this is because the learning process in this model aims at updating the network parameters for the output to be close to the ground truth without extracting any features from the input data. The huge number of parameters in this model (around 9 million) is another drawback. The training time is moderate (7.3 min) due to network simplicity.

By introducing the DCNN as a front-end, around 10% enhancement in the accuracy, sensitivity and specificity is achieved and the false alarm rate is improved by 60%. This improvement is due to the ability of DCNN to extract the spatial features across different scalp positions to use it in discrimination between preictal and interictal brain states. On the other hand, the training time is increased by 5 min and this is due to the added computation complexity by the DCNN. The network parameters are drastically decreased because of the parameter sharing and sparse connectivity properties of the DCNN. In the third model, Bi-LSTM is used as the back-end along with DCNN and this model increase the accuracy to be 99.6%, the sensitivity to be 99.72% and the specificity to be 99.6%. The false alarm rate is enhanced a lot to reach 0.004 false alarm per hour. This improvement is due to using Bi-LSTM as a classifier instead of MLP. Bi-LSTM extracts temporal features from the input sequence which helps in seizure prediction more accurately at the cost of training time which reached 14.2 min. The number of parameters is decreased by 94% by getting rid of the MLP. In the fourth model, DCAE is used to train the front-end part of the model. This improves the network optimization by starting the training with an initial set of parameters that makes the convergence process faster. As a result, the training time decreased to 4.25 min on average with the same highest performance. Utilizing the transfer learning technique reduces overfitting and generalizes better.

TABLE 2

| | | | | | Training | |
|---|---|---|---|---|---|---|
| Claimed | | | | False | Time | No. of |
| Model | Sensitivity | Specificity | Accuracy | Alarm h⁻¹ | (min) | Parameters |
| MLP | 84.67% | 82.60% | 83.63% | 0.174 | 7.3 | 8,870,291 |
| DCNN + | 95.41% | 92.80% | 94.10% | 0.072 | 12.5 | 520,477 |
| MLP | | | | | | |
| DCNN + | 99.72% | 99.60% | 99.66% | 0.004 | 14.2 | 27,657 |
| Bi-LSTM | | | | | | |
| DCAE + | 99.72% | 99.60% | 99.66% | 0.004 | 14.25 | 27,657 |
| Bi-LSTM | | | | | | |
| DCAE + | 99.72% | 99.60% | 99.66% | 0.004 | 2.2 | 18,345 |
| Bi-LSTM + | | | | | | |
| CS | | | | | | |

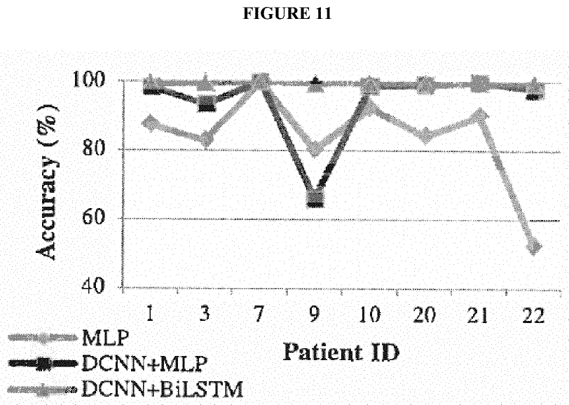

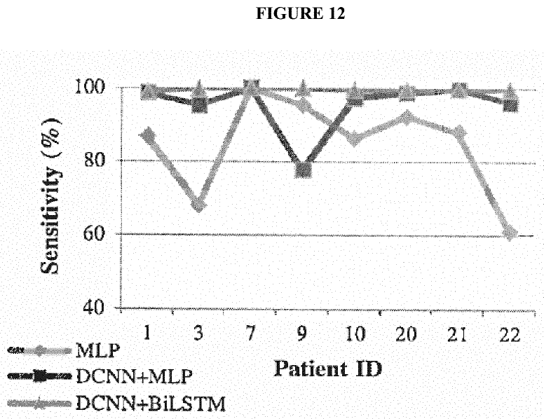

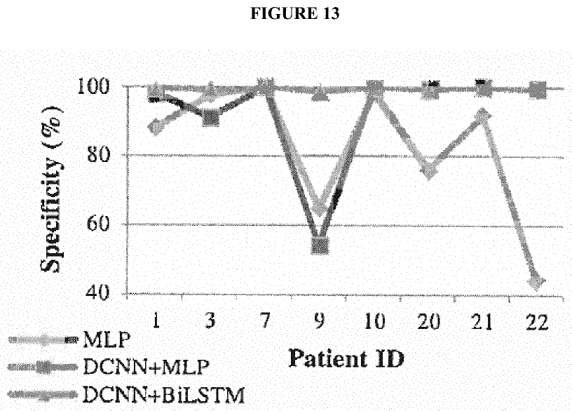

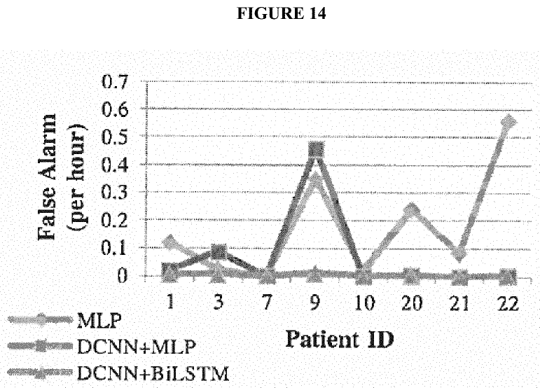

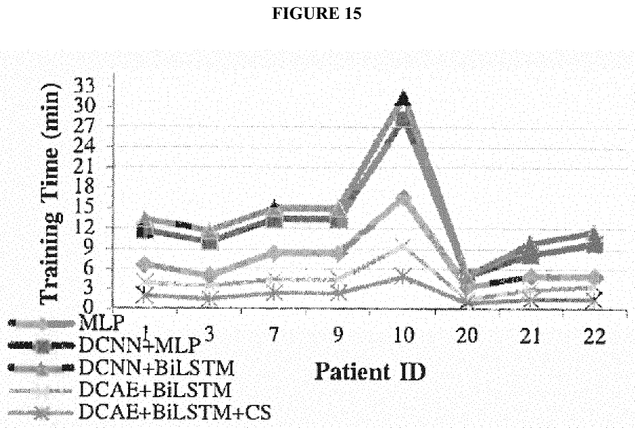

The channel selection algorithm shown at FIG. 17 reduces the number of channels to 10 channels on average among all the selected patients instead of using all the channels which are 23 channels. Therefore, the computation complexity is reduced making the training time to reach 2.2 min on average with lowest number of parameters of around 18K which make this model suitable for real-time applications. All the obtained results are shown graphically for different models across the selected patients in (FIGS. 11-15).

Regarding the prediction time, all the claimed models were able to accurately predict the tested seizures from the start of the preictal segments, thus the prediction time is one hour before the seizure onset or less in case of a shorter preictal segment.

The Kruskal-Wallis test is performed as a nonparametric test statistic to compare the accuracy, sensitivity, specificity and false alarm rate of each model of the three basic models which are, MLP, DCNN+MLP, and DCNN+Bi-LSTM. The Kruskal-Wallis test yielded (p-value<0.05) for all the performance measures indicating statistical significance difference between the results among all the claimed models. For the accuracy (p-value=0.01), for the sensitivity (p-value=0.006), for the specificity (p-value=0.04), and for the false alarm rate (p-value=0.04).

TABLE 3

| Method | | | | | | False | | |
|---|---|---|---|---|---|---|---|---|
| Feature | | Data | | | | Alarm | Predication | |
| Extraction | Classification | Selection | Sensitivity | Specificity | Accuracy | h⁻¹ | Time | |
| ZC Interval | GMM | LPOCV | 83.81% | NA | NA | 0.165 | 19.8 | min |
| ZC in WT | SVM | LPOCV | 96% | 90% | 94% | NA | NA | |
| Time, Freq., | SVM | 10-fold CV | 85.75% | 85.75% | 85.75% | NA | NA | |
| Graph | | | | | | | | |
| WT | CNN | 10-fold CV | 87.8% | NA | NA | 0.147 | 5.8 | min |
| Spectral | SVM | LOOCV | 98.68% | NA | NA | 0.046 | 42.7 | min |
| Power | | | | | | | | |

| | | | | | | | |
|---|---|---|---|---|---|---|---|
| DCAE + Bi-LSTM | LOOCV | 99.72% | 99.60% | 99.66% | 0.004 | 1 | hr. |

For further evaluation of the method, the achieved experimental results are compared with prior art that have used the same dataset as shown in Table 3. In the presented prior art, some features were extracted like Zero-Crossing (ZC) interval in the EEG signals as in the first compared method and ZC of the Wavelet Transform (WT) coefficients of the EEG signals as in the second compared method, WT of the EEG signals as in the fourth compared method, spectral power as in the fifth compared method and set of features in time domain, frequency domain and from graph theory as in the third compared method. These studies used machine learning based classifiers like SVM or Gaussian Mixture Model (GMM). The authors in the fourth compared method used CNN as a classifier. The claimed method achieved the highest accuracy, sensitivity and specificity among others. The prediction time is the earliest and the false alarm rate is the lowest.

A novel deep learning based patient-specific epileptic seizure prediction method using long-term scalp EEG data is provided. This method achieves a prediction accuracy of 99.6%, a sensitivity of 99.72%, a specificity of 99.60%, a false alarm rate of 0.004 per hour and prediction time of one hour prior the seizure onset. Important spatial and temporal features from raw data are learned by the DCNN and Bi-LSTM networks respectively. DCAE based Semi-supervised learning approach is investigated with the transfer learning technique which led to reducing the training time. For the system to be suitable for real-time application, a channel selection algorithm is shown at FIG. 17, which reduces the computational load and the training time. Using Leave-One-Out exhaustive cross-validation technique to test the claimed models proves the robustness and generality of the method against variation across various seizure types.

The experimental results and the comparison with prior art demonstrate that the method is efficient, reliable and suitable for real-time application of seizure prediction. This is by achieving accuracy higher than the state of the art with earlier prediction time to mitigate the potential life-threatening incidents for epileptic patients.

### EXAMPLE 2

Hardware implementation of deep neural networks faces a lot of challenges due to the computation complexity and high demand for resources like memory and computation units. In one embodiment, the inference network of the inventive model is implemented on FPGA. In the implementation, the FPGA parallelism is used to speed up the computation. This means that external storage to store the network parameters does not need to be accessed. And, therefore, an 8-bit precision can be used to represent both the data and the parameters.

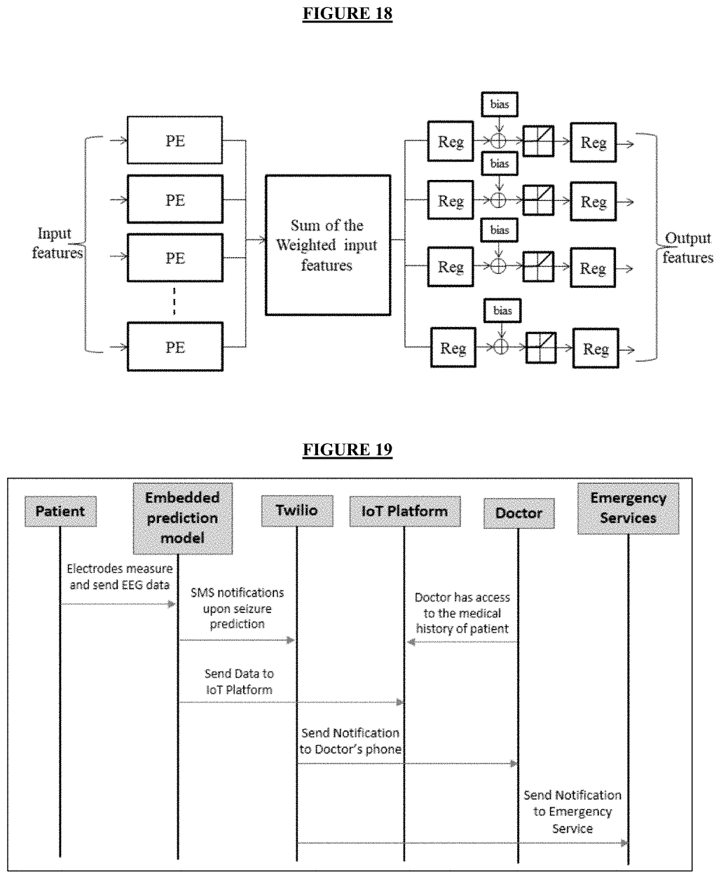

The implemented model utilizes the DCNN only for feature extraction and classification. As the inference network of the claimed model consists of convolutional, pooling and fully connected layers, three main modules for each type of layer are implemented. FIG. 18 illustrates the convolution module implementation. As could be seen from FIG. 18, the convolution module comprises three main units which are the processing elements (PEs), adder unit and output unit. The PEs execute the convolution operation between the input vectors and the kernels. In each PE, one input feature map is multiplied with all the kernels' weights in the same clock cycle by shifting the feature map through a set of registers with the same size of the kernels. After weight multiplication, the resultant values are added up to form the corresponding output feature maps. All the PEs are working in parallel and their outputs are added up in the adder unit of the convolution module and then added to the corresponding bias and passed through ReLU to construct the output feature maps as shown in FIG. 18.

The second module (pooling) takes the output features and applies maxpooling operation on them through PEs only. Each PE is applied to one feature map to select the largest value in a specific vector length by using comparators. Pooling is performed on all the feature maps in the same clock cycle to form the final layer feature maps. Finally, the fully connected module is the same as the convolution module except that the PE performs matrix multiplication instead of the convolution operation. All network parameters are hardcoded rather than stored in an external memory due to the restrictions on the precision and the number of kernels imposed.

The following provides an example of the type of equipment used to implement the system. Any suitable equipment may be used. The PYNQ-Z2 FPGA board was used, based on Xilinx Zyng™ SoC to implement the architecture. For behavior evaluation, the ModelSim PE 10.4a was used as a simulator. Synthesis and implementation are done via Vivado 2019.1 tool using Very High Speed Integrated Circuit Hardware Description Language (“VHDL”).

### EXAMPLE 3

An IoT cloud-based platform is shown in FIG. 19. The EEG signals are measured using an EEG headset that sends the recording to the FPGA via Bluetooth. The FPGA implements the claimed prediction model in Example 2. The EEG recording and the prediction result are sent to the processor, which is considered the network gateway. The continuous EEG recording is uploaded to the cloud for monitoring and history evaluation by the doctor. In the case of the model detects an incoming seizure, it raises an alarm to the patient and sends a notification to the doctor and any emergency service to protect the patient from any potential accident. The system's components are given in details as following:

EEG monitoring headset: The EEG electrodes are placed on a headset that offers Bluetooth capabilities to send electrodes' readings as an input to the pre-trained model for seizure prediction.

Deep learning model: The claimed seizure prediction model is embedded on FPGA to facilitate edge computing. The PYNQ-Z2 FPGA board was used, based on Xilinx Zyng™ SoC with ethernet cores and running a client socket program used to send predictions to a processor (for example, a Raspberry Pi).

Architecture gateway: The architecture utilizes a Raspberry Pi 3+ device with wireless fidelity (“WiFi”) capabilities to send data to a cloud-based platform. However, any suitable processor may be used. This provides the device the convenience to easily connect to the cloud-based IoT platform. The Raspberry Pi module is connected to the FPGA via an ethernet connection.

Simple Message Service (“SMS”) Notification: This notification system is utilized to send SMS to the doctor and emergency services in the event where the model predicts seizure. Emergency service is an important option because it can call the patient and based on responses received, a healthcare provider may dispatch an ambulance to the patient's location. In one embodiment of the architecture, the Twilio application programming interface is used to send SMS to doctor and emergency services. Twilio is a cloud-based application that allows software developers to programmatically send and receive text messages using its web service APIs. Any such application may be used.

IoT Platform: The platform is used to store the patient's historical seizure predictions and recording. In one embodiment of the architecture, an open-source IoT platform is used because all of the analysis and calculations are carried out in the patient's device. One example of an open-source IoT platform is ThingsBoard. However, any open-source IoT platform that offers services such as, device management, data collection, processing and visualization for IoT projects may be used.

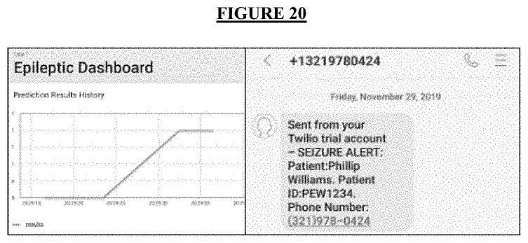

One embodiment of the claimed IoT-based network protocol is illustrated in FIG. 19 showing all the above components and their connections. An example of the received SMS notification is shown in FIG. 20 (left side). The SMS contains the patient ID and patient name. The doctor can view the historical and current data on the status of the patient. The doctor can use the functionalities on the platform to display data received in a graphical manner, such as a line graph of the time predictions were recorded versus the predictions as shown in FIG. 20 (right side).

An evaluation was conducted for the claimed prediction algorithm on the selected subjects from CHB-MIT dataset. Some performance metrics are calculated like accuracy, sensitivity, and specificity. The result is calculated on the test set after training the model on the EEG data from 10 channels instead of the entire 23 channels. The selected channels are the most important ones claimed by the channel selection algorithm. The computation complexity is reduced due to the lower number of channels involved in the experiment allowing the real-time operation for the claimed IoT framework. The obtained results are: prediction accuracy is 96.1%, sensitivity is 97.41%, and specificity is 94.8%.

The achieved results show that the claimed method is reliable, efficient, and suitable for real-time application to predict epileptic seizures. The high prediction accuracy of 96.1% with lower complexity and smaller memory footprint make the claimed system a good choice for a smart healthcare system to improve the quality of life for epileptic patients.

For the purpose of understanding the SYSTEM AND METHOD FOR EARLY AND EFFICIENT PREDICTION OF EPILECTIC SEIZURES, references are made in the text to exemplary embodiments of a SYSTEM AND METHOD FOR EARLY AND EFFICIENT PREDICTION OF EPILECTIC SEIZURES, only some of which are described herein. It should be understood that no limitations on the scope of the invention are intended by describing these exemplary embodiments. One of ordinary skill in the art will readily appreciate that alternate but functionally equivalent components, materials, designs, and equipment may be used. The inclusion of additional elements may be deemed readily apparent and obvious to one of ordinary skill in the art. Specific elements disclosed herein are not to be interpreted as limiting, but rather as a basis for the claims and as a representative basis for teaching one of ordinary skill in the art to employ the present invention.

1. A patient-specific epileptic seizure prediction method comprising deep learning based algorithms, wherein said prediction method does not comprise preprocessing of scalp electroencephalogram recordings.

2. The method of claim 1 further comprising classification tasks, and wherein said classification tasks comprise the step of applying an artificial neural network to raw electroencephalogram recordings as a classifier.

3. The method of claim 2 wherein said artificial neural network is Multi-layer Perceptron.

4. The method of claim 2 wherein said classification tasks further comprise the step of applying to said raw electroencephalogram recordings a Deep Convolutional Neural Network to learn the discriminative spatial features between interictal and preictal brain states before said artificial neural network is applied for said classification.

5. The method of claim 1 further comprising classification tasks, wherein said classification tasks comprise concatenating a Bidirectional Long Short-Term Memory Recurrent Neural Network to a Deep Convolutional Neural Network.

6. The method of claim 5 wherein said classification tasks further comprise the step of applying a pretrained encoder.

7. The method of claim 6 wherein said pretrained encoder is a part of an Autoencoder based semi-supervised model.

8. An epileptic seizure prediction method comprising a channel selection algorithm, wherein said channel selection algorithm outputs representative channels from a multi-channel electroencephalogram recording.

9. The method of claim 1 wherein said deep learning based algorithm is embedded on a field-programmable gate array and wherein said scalp electroencephalogram recordings are transmitted to said field-programmable gate array and said algorithm is applied to produce a continuous seizure predication reading.

10. The method of claim 9 wherein said continuous seizure predication reading is available on a cloud.

11. The method of claim 10 further comprising an alarm feature wherein when said continuous seizure prediction reading signals an upcoming seizure, a notification is transmitted to a designated person.
